(19) United States
(12) Patent Application Publication
(10) Pub. No.: US 2005/0009066 A1
(43) Pub. Date: Jan. 13, 2005

(54) Methods for localizing target molecules in a flowing fluid sample

(76) Inventor: Dennis M. Connolly, Rochester, NY (US)

Correspondence Address: Michael L. Goldman;Nixon Peabody LLP, Clinton Square, P.O. Box 31051, Rochester, NY, 14603-1051, US

(21) Appl. No.: 10/847,196

(22) Filed: May 17, 2004

Related U.S. Application Data
(60) Provisional application No. 60/470,645, filed on May 15, 2003.

(51) Int. Cl. C12Q 1/68; C12M 1/34; G01N 33/558

(52) U.S. Cl. 435/6; 435/287.2; 436/514

(57) ABSTRACT

The present invention relates to a device and method for concentrating and detecting target molecules in a flowing fluid sample. The device includes a housing defining a passage through which a fluid sample flows and a concentrating device positioned in the housing and including at least one electrode on a first side of the passage. The at least one electrode on the first side of the passage has a polarity which electrostatically attracts target molecules in the flowing fluid sample. The device also includes a detection device downstream of the concentrating device and including test structures having capture probes that are capable of specifically binding to the target molecules, if any, in the flowing fluid sample. The test structures are positioned in the housing on the first side of the passage.

This application claims the benefit of U.S. Provisional Patent Application Ser. No. 60/470,645, filed May 15, 2003, which is hereby incorporated by reference in its entirety.

### FIELD OF THE INVENTION

The present invention relates to devices and methods for concentrating and detecting target molecules in a flowing fluid sample.

### BACKGROUND OF THE INVENTION

Nucleic acids, such as DNA or RNA, have become of increasing interest as analytes for clinical or forensic uses. Powerful new molecular biology technologies enable one to detect congenital or infectious diseases. These same technologies can characterize DNA for use in settling factual issues in legal proceedings, such as paternity suits and criminal prosecutions.

For the analysis and testing of nucleic acid molecules, amplification of a small amount of nucleic acid molecules, isolation of the amplified nucleic acid fragments, and other procedures are necessary. The science of amplifying small amounts of DNA have progressed rapidly and several methods now exist. These include linked linear amplification, ligation-based amplification, transcription-based amplification and linear isothermal amplification. Linked linear amplification is described in detail in U.S. Pat. No. 6,027,923 to Wallace et al. Ligation-based amplification includes the ligation amplification reaction (LAR) described in detail in Wu et al., Genomics, 4:560 (1989) and the ligase chain reaction described in European Patent No. 0320308B1. Transcription-based amplification methods are described in detail in U.S. Pat. Nos. 5,766,849 and 5,654,142, Kwoh et al., Proc. Natl. Acad. Sci. U.S.A., 86:1173 (1989), and PCT Publication No. WO 88/10315 to Ginergeras et al. The more recent method of linear isothermal amplification is described in U.S. Pat. No. 6,251,639 to Kurn.

The most common method of amplifying DNA is by the polymerase chain reaction (“PCR”), described in detail by Mullis et al., Cold Spring Harbor Quant. Biol., 51:263-273 (1986), European Patent No. 201,184 to Mullis, U.S. Pat. No. 4,582,788 to Mullis et al., European Patent Nos. 50,424, 84,796, 258017, and 237362 to Erlich et al., and U.S. Pat. No. 4,683,194 to Saiki et al. The PCR reaction is based on multiple cycles of hybridization and nucleic acid synthesis and denaturation in which an extremely small number of nucleic acid molecules or fragments can be multiplied by several orders of magnitude to provide detectable amounts of material. One of ordinary skill in the art knows that the effectiveness and reproducibility of PCR amplification is dependent, in part, on the purity and amount of the DNA template. Certain molecules present in biological sources of nucleic acids are known to stop or inhibit PCR amplification (Belec et al., Muscle and Nerve 21(8):1064 (1998); Wiedbrauk et al., Journal of Clinical Microbiology, 33(10):2643-6 (1995); Deneer and Knight, Clinical Chemistry 40(1):171-2 (1994)). For example, in whole blood, hemoglobin, lactoferrin, and immunoglobulin G are known to interfere with several DNA polymerases used to perform PCR reactions (Al-Soud and Radstrom, Journal of Clinical Microbiology, 39(2):485-493 (2001); Al-Soud et al., Journal of Clinical Microbiology, 38(1):345-50 (2000)). These inhibitory effects can be more or less overcome by the addition of certain protein agents, but these agents must be added in addition to the multiple components already used to perform the PCR. Thus, the removal or inactivation of such inhibitors is an important factor in amplifying DNA from select samples.

On the other hand, isolation and detection of particular nucleic acid molecules in a mixture requires a nucleic acid sequencer and fragment analyzer, in which gel electrophoresis and fluorescence detection are combined. Unfortunately, electrophoresis becomes very labor-intensive as the number of samples or test items increases.

For this reason, a simpler method of analysis using DNA oligonucleotide probes is becoming popular. New technology, called VLSIPS™, has enabled the production of chips smaller than a thumbnail where each chip contains hundreds of thousands or more different molecular probes. These techniques are described in U.S. Pat. No. 5,143,854 to Pirrung et al., PCT Publication No. WO 92/10092, and PCT WO 90/15070. These biological chips have molecular probes arranged in arrays where each probe ensemble is assigned a specific location. These molecular array chips have been produced in which each probe location has a center to center distance measured on the micron scale. Use of these array type chips has the advantage that only a small amount of sample is required, and a diverse number of probe sequences can be used simultaneously. Array chips have been useful in a number of different types of scientific applications, including measuring gene expression levels, identification of single nucleotide polymorphisms, and molecular diagnostics and sequencing as described in U.S. Pat. No. 5,143,854 to Pirrung et al.

Array chips where the probes are nucleic acid molecules have been increasingly useful for detection for the presence of specific DNA sequences. Most technologies related to array chips involve the coupling of a probe of known sequence to a substrate that can either be structural or conductive in nature. Structural types of array chips usually involve providing a platform where probe molecules can be constructed base by base or covalently binding a completed molecule. Typical array chips involve amplification of the target nucleic acid followed by detection with a fluorescent label to determine whether target nucleic acid molecules hybridize with any of the oligonucleotide probes on the chip. After exposing the array to a sample containing target nucleic acid molecules under selected test conditions, scanning devices can examine each location in the array and quantitate the amount of hybridized material at that location. Alternatively, conductive types of array chips contain probe sequences linked to conductive materials such as metals. Hybridization of a target nucleic acid typically elicits an electrical signal that is carried to the conductive electrode and then analyzed.

For most solid support or array technologies, small oligonucleotide capture probes are immobilized or synthesized on the support. The sequence of the capture probes imparts the specificity for the hybridization reaction. Several different chemical compositions exist currently for capture probe studies. The standard for many years has been straight deoxyribonucleic acids. The advantage of these short single stranded DNA molecules is that the technology has existed for many years and the synthesis reaction is relatively inexpensive. Furthermore, a large body of technical studies is available for quick reference for a variety of scientific techniques, including hybridization. However, many different types of DNA analogs are now being synthesized commercially that have advantages over DNA oligonucleotides for hybridization. Some of these include PNA (protein nucleic acid), LNA (locked nucleic acid) and methyl phosphonate chemistries. In general, all of the DNA analogs have higher melting temperatures than standard DNA oligonucleotides and can more easily distinguish between a fully complementary and single base mis-match target. This is possible because the DNA analogs do not have a negatively charged backbone, as is the case with standard DNA. This allows for the incoming strand of target DNA to bind tighter to the DNA analog because only one strand is negatively charged. The most studied of these analogs for hybridization techniques is the PNA analog, which is composed of a protein backbone with substituted nucleobases for the amino acid side chains (see www.appliedbiosystems.com or www.eurogentec.com). Indeed, PNAs have been used in place of standard DNA for almost all molecular biology techniques including DNA sequencing (Arlinghaus et al., Anal Chem., 69:3747-53 (1997)), DNA fingerprinting (Guerasimova et al., Biotechniques 31:490-495 (2001)), diagnostic biochips (Prix et al., Clin. Chem., 48:428-35 (2002); Feriotto et al., Lab Invest, 81:1415-1427 (2001)), and hybridization based microarray analysis (Weiler et al., Nucleic Acids Res, 25:2792-2799 (1997); Igloi, Genomics, 74:402-407 (2001)).

Techniques for forming sequences on a substrate are known. For example, the sequences may be formed according to the techniques disclosed in U.S. Pat. No. 5,143,854 to Pirrung et al., PCT Publication No. WO 92/10092, or U.S. Pat. No. 5,571,639 to Hubbell et al. Although there are several references on the attachment of biologically useful molecules to electrically insulating surfaces such as glass (http://www.piercenet.com/Technical/default.cftn?tmpl=../Lib/ViewDoc.cfm&doc=3483; McGovern et al., Langmuir, 10:3607-3614 (1994)) or silicon oxide (Examples 4-6 of U.S. Pat. No. 6,159,695 to McGovern et al.), there are few examples of effective molecular attachment to electrically conducting surfaces except for gold (Bain et al., Langmuir, 5:723-727 (1989)) and silver (Xia et al., Langmuir 22:269, (1998)). In general, the problem of attaching biologically active molecules to the surface of a substrate, whether it is a metal electrical conductor or an electrical insulator such as glass, is more difficult than the simple chemical reaction of a reactive group on the biological molecule with a complementary reactive group on the substrate. For example, a metal electrical conductor has no reactive sites, in principle, except those that may be adventitiously or deliberately positioned on the surface of the metal.

Hybridization of target DNAs to such surface bound capture probes poses difficulties not seen, if both species are soluble. Steric effects result from the solid support itself and from too high of a probe density. Studies have shown that hybridization efficiency can be altered by the insertion of a linker moiety that raises the complementary region of the probe away from the surface (Schepinov et al., Nucleic Acid Res., 25:1155-1161 (1997); Day et al., Biochem J., 278:735-740 (1991)), the density at which probes are deposited (Peterson et al., Nucleic Acids Res., 29:5163-5168 (2001); Wilkins et al., Nucleic Acids Res., 27:1719-1729)), and probe conformation (Riccelli et al., Nucleic Acids Res., 29:996-1004 (2001)). Insertion of a linker moiety between the complementary region of a probe and its attachment point can increase hybridization efficiency and optimal hybridization efficiency has been reported for linkers between 30 and 60 atoms in length. Likewise, studies of probe density suggest that there is an optimum probe density, and that this density is less than the total saturation of the surface (Schepinov et al., Nucleic Acid Res., 25:1155-1161 (1997); Peterson et al., Nucleic Acids Res., 29:5163-5168 (2001); Steel et al., Anal. Chem., 70:4670-4677 (1998)). For example, Peterson et al. reported that hybridization efficiency decreased from 95% to 15% with probe densities of 2.0×10¹² molecules/cm² and 12.0×10¹² molecules/cm², respectively.

Quantitiation of hybridization events often depends on the type of signal generated from the hybridization reaction. The most common analysis technique is fluorescent emission from several different types of dyes and fluorophores. However, quantitating samples in this manner usually requires a large amount of the signaling molecule to be present to generate enough emission to be quantitated accurately. More importantly, quantitation of fluorescence generally requires expensive analysis equipment for linear response. Furthermore, the hybridization reactions take up to two hours, which for many uses, such as detecting biological warfare agents, is simply too long. Therefore, a need exists for a system which can rapidly detect biological material in samples.

Recently, new approaches for electronic DNA detection have been developed which can detect even single copies of nucleic acid molecules and can also be applied to other biological molecules such as proteins or whole cells. These approaches overcome the limitations of PCR-based technologies and can be readily automated for reliable field use. The sensitivity of the electronic based detection system described in U.S. patent application Ser. No. 10/288,657 is not limited by the signal strength of the sensor system but rather by the rate of hybridization of target nucleic acid molecules to capture probes on the test structures.

The rate of DNA hybridization is directly proportional to the concentration of the target DNA molecules. Moreover, the accessibility of DNA for hybridization is diffusion limited. Modeling clearly demonstrates that areas immediate to bound probes become depleted for target and the rate of hybridization is then limited by the diffusion of other target DNA molecules to the area near the probes. Furthermore, for small volume flow cells, laminar flow dominates. Mixing of the target DNA within the flow cell is very limited, making it very difficult to compensate for limited diffusion by mixing.

Electronic fields have been used for years to move nucleic acid molecules and even to concentrate nucleic acid molecules. However, electronic sensors for DNA detection can not be directly charged without breaking down the sensor. Positively charging a gold electrode results in the breakdown of the gold surface and removal of attached capture probes. Furthermore, charge located immediate to the sensor can limit the diffusion of the target molecules and, thus, inhibit free interaction with the capture molecules. Therefore, a need exists for a device and a method which can concentrate target molecules near test sites containing capture probes on a surface.

The present invention is directed to achieving these objectives.

### SUMMARY OF THE INVENTION

The present invention relates to a device for concentrating and detecting target molecules in a flowing fluid sample. The device includes a housing defining a passage through which a fluid sample flows and a concentrating device positioned in the housing and including at least one electrode on a first side of the passage. The at least one electrode on the first side of the passage has a polarity which electrostatically attracts target molecules in the flowing fluid sample. The device also includes a detection device downstream of the concentrating device and including test structures having capture probes that are capable of specifically binding to the target molecules, if any, in the flowing fluid sample. The test structures are positioned in the housing on the first side of the passage.

Another aspect of the present invention relates to a method for concentrating and detecting target molecules in a flowing fluid sample. The method first involves providing a device including a housing defining a passage through which a fluid sample flows and a concentrating device positioned in the housing and including at least one electrode on a first side of the passage. The at least one electrode on the first side of the passage has a polarity which electrostatically attracts target molecules in the flowing fluid sample. The device also includes a detection device downstream of the concentrating device and comprising test structures having capture probes that are capable of specifically binding to the target molecules, if any, in the flowing fluid sample. The test structures are positioned in the housing on the first side of the passage. Next, the method involves introducing a sample containing target molecules into the device. Then, an electric field is applied to the at least one electrode on the first side of the passage of the concentrating device under conditions effective to electrostatically attract and concentrate the target molecules, if any, in the flowing fluid sample near the at least one electrode on the first side of the passage of the concentrating device. Finally, the concentrated target molecules in the flowing fluid sample is permitted to specifically bind to the capture probes of the detection device, where the presence of the target molecules in the flowing fluid sample is detected.

The present invention provides a method for concentrating biological molecules in a flowing fluid sample to an area of the flow which can be directed to where capture probes will be located. The present invention utilizes laminar flow and the limited diffusion of the charged molecules to keep the charged molecules in an area near the bound capture molecules after they have been focused by use of an electronic field. The present invention is simple to manufacture and does not interfere with the attachment of the target molecules to the surface of the detector or the stability of the detector itself.

### BRIEF DESCRIPTION OF THE DRAWINGS

FIG. 1 provides side (a) and top (b) views of the device of the present invention.

FIG. 2(a) shows field lines and FIG. 2(b) shows potential concentration gradients for two different embodiments of the device of the present invention. In one embodiment, the positive terminal is smaller than the negative terminal, the field lines converge and the concentration is in a more compact area.

FIG. 3 shows nucleic acid molecule concentration gradients from a side view (a) and a top view (b).

FIG. 4(a) shows how nucleic acid molecules are concentrated. Laminar flow of the fluids places the concentrated nucleic acid molecules over the test structures as shown in FIG. 4(b). The laminar flow may be reversed in order to run the concentrated nucleic acid molecules over the test structures from the other direction. The field lines shown in FIG. 4(c) may be used in two ways. They can be used to move the nucleic acid molecules back and forth across the test structures by switching the polarity of the terminals. Alternatively, they can be used to remove any non-complementary nucleic acid molecules from the chamber, along with the laminar flow of the fluid.

FIG. 5 shows the equations for calculating the diffusivity and mobility for DNA, which can be used to model the system of the present invention.

FIGS. 6(a) and 6(b) illustrate the change in DNA concentration in a model system of the present invention versus the position of the DNA molecules in two different dimensions, X and Y, respectively, when different electric potentials (U) are applied for different periods of time (T).

FIG. 7 is a schematic drawing showing one embodiment of the detection device with a plurality of different groups of electrically separated electrical conductors for detection of target nucleic acid molecules.

FIG. 8 is a schematic drawing showing a second embodiment of the detection device with a plurality of different groups of electrically separated electrical conductors for detection of target nucleic acid molecules.

FIG. 9 is a schematic drawing showing a third embodiment of the detection device with a plurality of different groups of electrically separated electrical conductors for detection of target nucleic acid molecules.

FIG. 10 is a schematic drawing showing a fourth embodiment of the detection device with a plurality of different groups of electrically separated electrical conductors for detection of target nucleic acid molecules.

FIG. 11 is a schematic drawing of a DNA concentrator card including the detection device of the present invention.

### DETAILED DESCRIPTION OF THE INVENTION

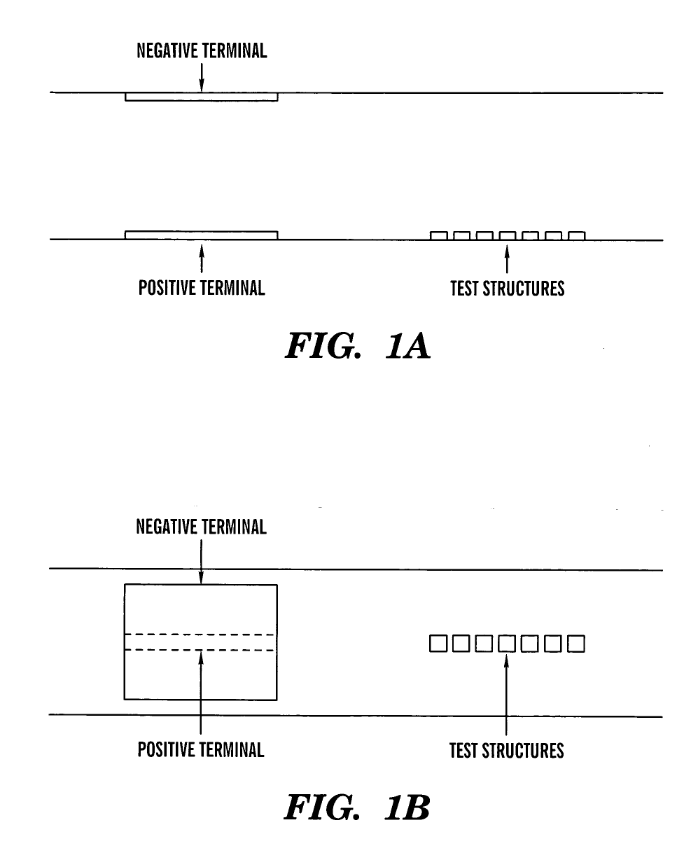

The present invention relates to a device for concentrating and detecting target molecules in a flowing fluid sample. The device includes a housing defining a passage through which a fluid sample flows and a concentrating device positioned in the housing and including at least one electrode on a first side of the passage. The at least one electrode on the first side of the passage has a polarity which electrostatically attracts target molecules in the flowing fluid sample. The device also includes a detection device downstream of the concentrating device and including test structures having capture probes that are capable of specifically binding to the target molecules, if any, in the flowing fluid sample. The test structures are positioned in the housing on the first side of the passage. FIG. 1 illustrates the device of the present invention.

Another aspect of the present invention relates to a method for concentrating and detecting target molecules in a flowing fluid sample. The method first involves providing a device including a housing defining a passage through which a fluid sample flows and a concentrating device positioned in the housing and including at least one electrode on a first side of the passage. The at least one electrode on the first side of the passage has a polarity which electrostatically attracts target molecules in the flowing fluid sample. The device also includes a detection device downstream of the concentrating device and comprising test structures having capture probes that are capable of specifically binding to the target molecules, if any, in the flowing fluid sample. The test structures are positioned in the housing on the first side of the passage. Next, the method involves introducing a sample containing target molecules into the device. Then, an electric field is applied to the at least one electrode on the first side of the passage of the concentrating device under conditions effective to electrostatically attract and concentrate the target molecules, if any, in the flowing fluid sample near the at least one electrode on the first side of the passage of the concentrating device. Finally, the concentrated target molecules in the flowing fluid sample is permitted to specifically bind to the capture probes of the detection device, where the presence of the target molecules in the flowing fluid sample is detected.

In one embodiment of the present invention, the target molecules are biological molecules. In particular, the biological molecules are proteins or nucleic acid molecules. In another embodiment of the present invention, the capture probes are antibodies. In other embodiments of the present invention, the capture probes are oligonucleotides or peptide nucleic acid analogs.

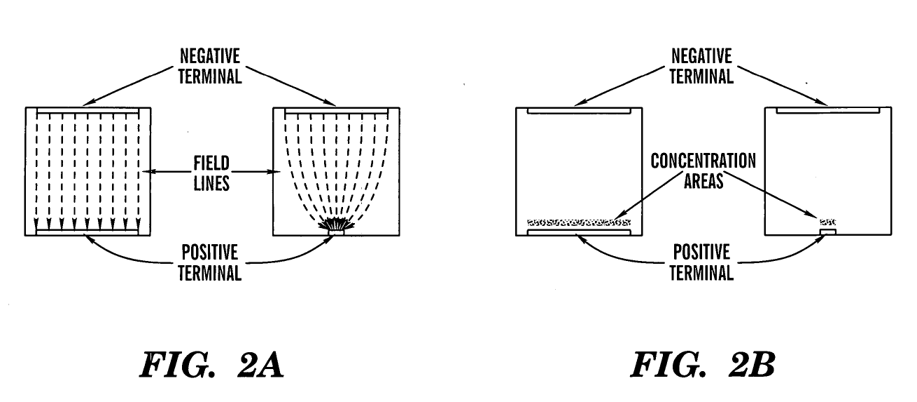

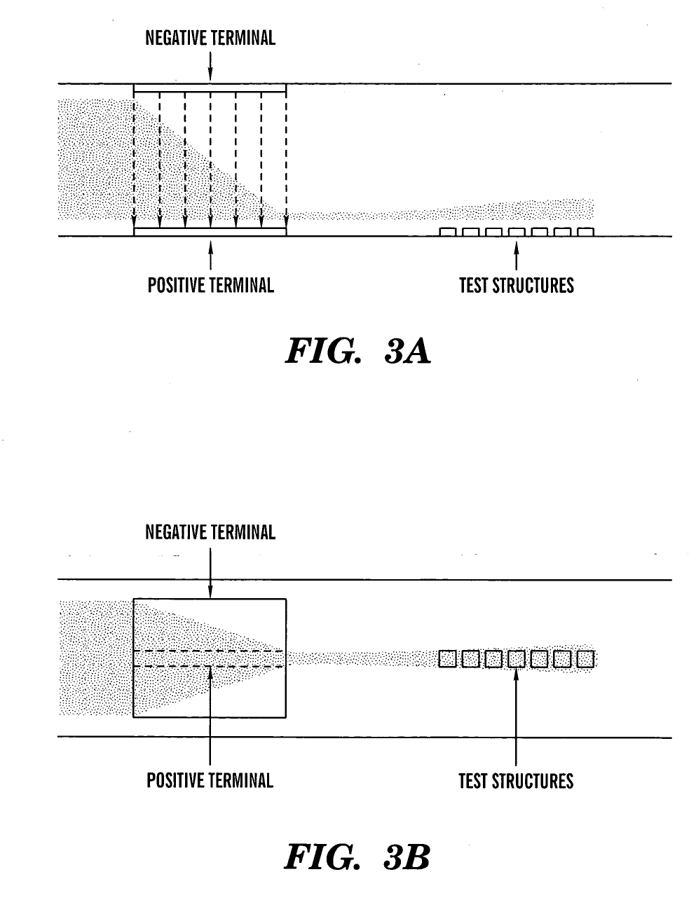

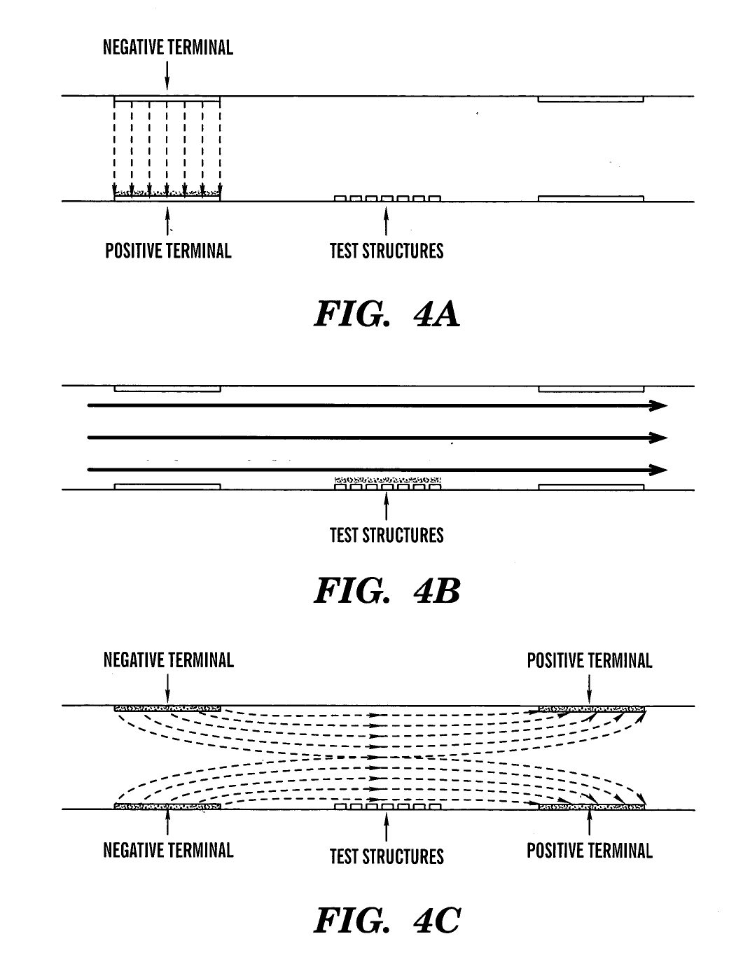

Charged molecules, such as DNA, can be moved in solution by application of an electric field. For example, DNA is a negatively charged molecule and will move away from a negatively charged electrode and towards a positively charged electrode. The present invention relies upon this phenomenon to concentrate target molecules in a flowing fluid sample. Thus, a solution containing the charged molecules is flowed between two oppositely charged electrodes. The charged molecules will then drift towards the positively charged electrode as shown in FIGS. 2-4. This results in a localized high concentration of the negatively charged molecule on the side of the device nearest the positive electrode.

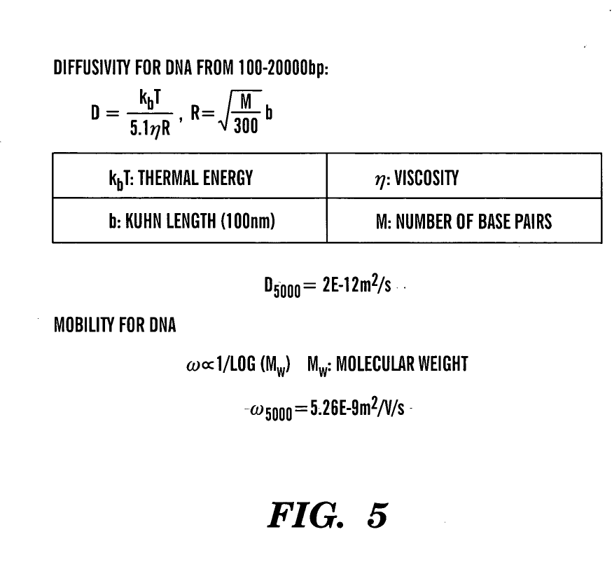

After the solution moves out of the concentrating device, the present invention relies upon laminar flow to create a virtual chamber. In other words, laminar flow will limit mixing of the material as it is transported to the surface bound capture probes. For larger molecules, such as proteins and nucleic acids, limited diffusion rates will further confine the charged molecules to a tight distribution within the flow. FIG. 5 shows the equations for calculating the diffusivity (D) and mobility (ω) for DNA, which can be used to model the system of the present invention.

In another embodiment of the present invention, the flow can be reversed by a flow reversing device so that the target molecules are made to flow back to the concentration device to reconcentrate the particles for repeated delivery to the test sites.

Alternatively, a reconcentrating device may be located downstream of the detection device and the flow can be reversed to move the target molecules back over the test sites to allow for multiple opportunities for hybridization to occur.

Multiple electrodes may also run along the base of the chamber, each of which can be independently charged. Such a design allows for each electrode to be used independently to focus target molecules for different sets of test sites arrays downstream of the different electrodes.

In another embodiment of the present invention, the electrodes on each side of the passage are different in size. For example, a wide negative electrode running parallel to the flowing fluid sample and a narrow positive electrode also running parallel to the flowing fluid sample will focus the target molecule such as DNA in two dimensions (right panels of FIGS. 2(a) and 2(b)).

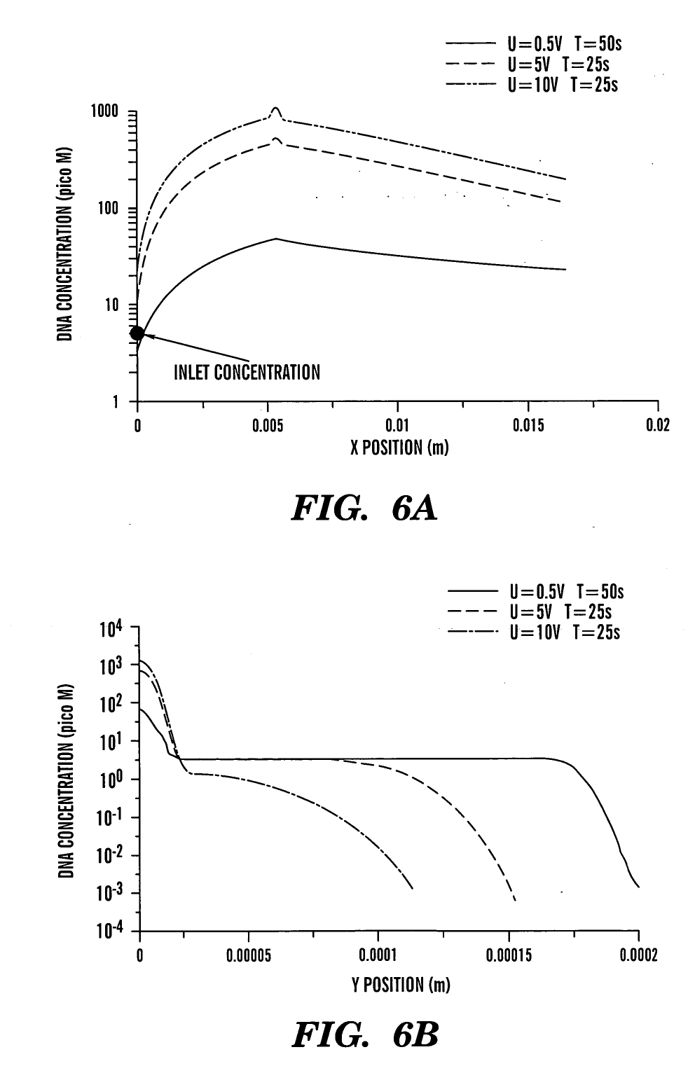

FIGS. 6A and 6B illustrate the change in DNA concentration in a model system of the present invention versus the position of the DNA molecules in two different dimensions, X and Y, respectively, when different electric potentials (U) are applied for different periods of time (T). For example, when an electric potential of 0.5V is applied for 50 seconds, DNA can be concentrated approximately 10 folds. When an electric potential of 5V is applied for 25 seconds, DNA can be concentrated approximately 100 folds. When an electric potential of 10V is applied for 25 seconds, DNA can be concentrated approximately 200 folds.

The flow of the fluid sample can be continuous or pulsed. Pulsed flow allows for free diffusion of the target molecules.

Since it has been observed that DNA and other molecules can react with exposed charged electrodes, it may be necessary to protect the electrodes of the concentrating device with a coating of material which prevents the target molecule from actually contacting the electrode but allowing the electrode electrical contact with the fluid flow.

Electric field properties and the charge of the target molecules can be modified by the makeup of the fluid used to move the target molecules. Thus, in other embodiments of the present invention, salts and pH can be used to facilitate or inhibit electrical mobility as needed.

Concentration of larger molecules may create layers within the flow having different viscosities. Thus, in another embodiment of the present invention, the variation in viscosity can be minimized by adding uncharged polymers to the fluid. These polymers would raise the overall viscosity but would not be affected directly by the application of the electric field to the fluid.

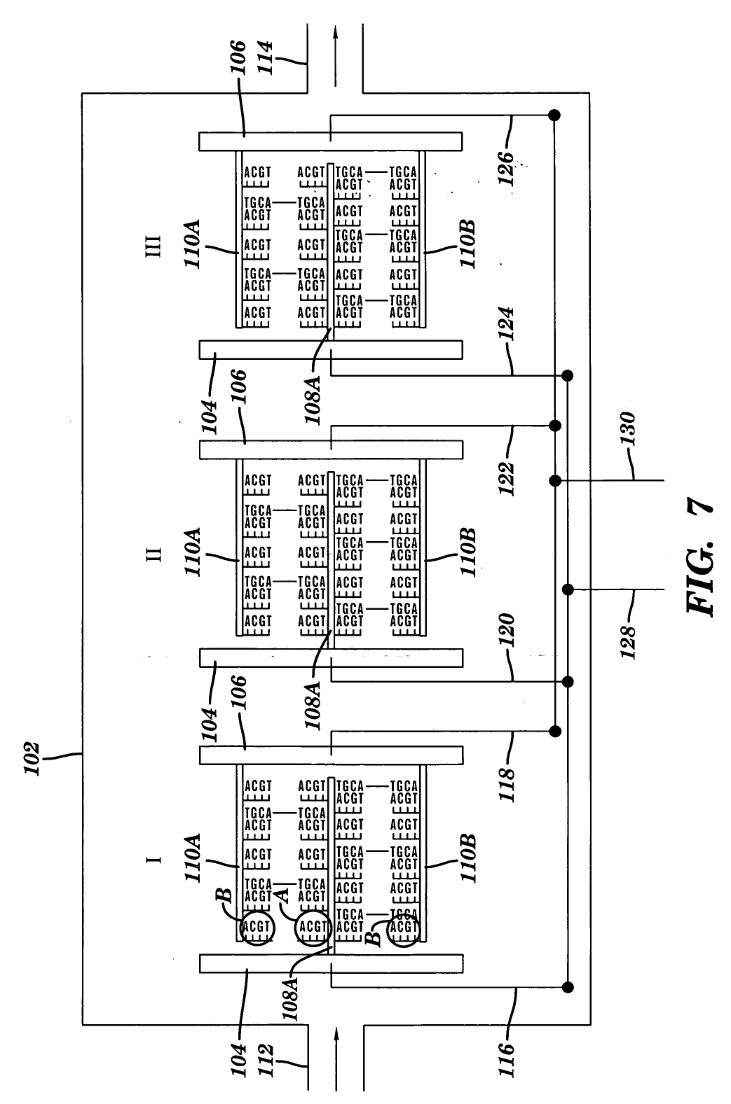

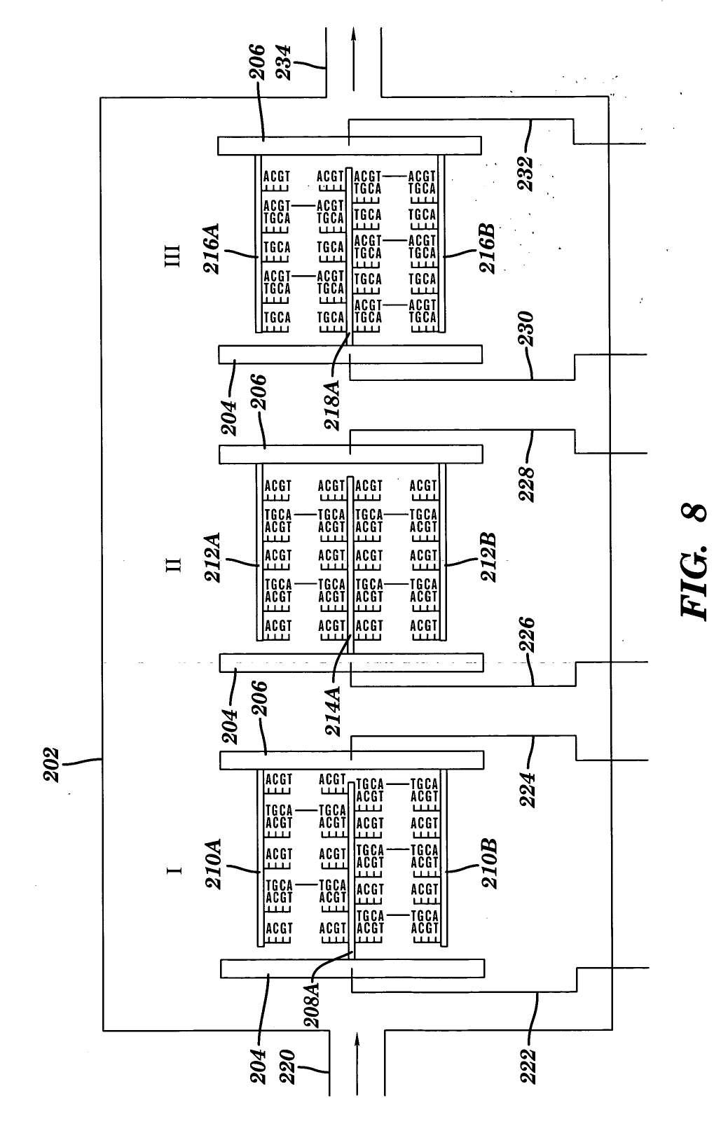

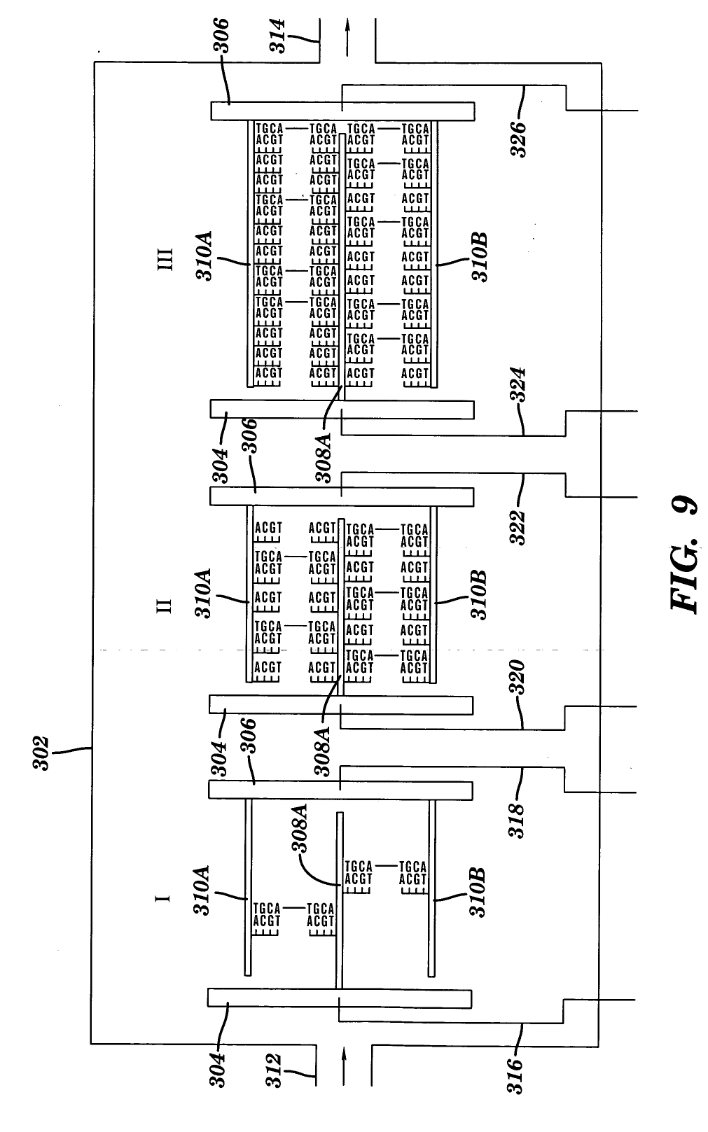

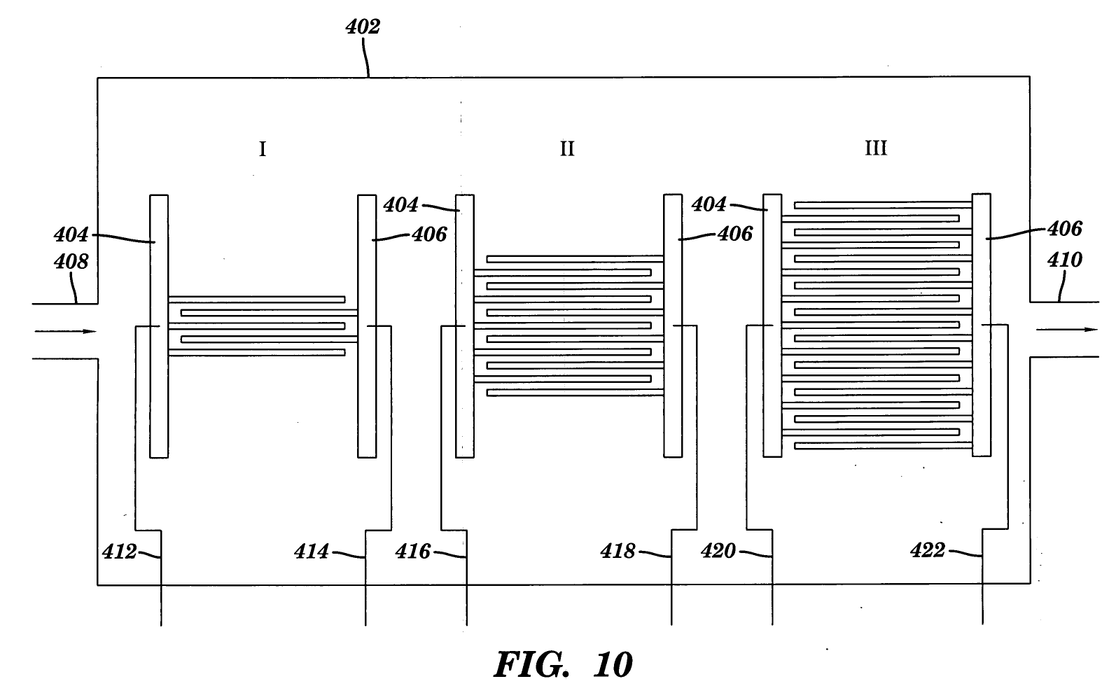

One aspect of the present invention involves the detection of multiple DNA sequences from a plurality of DNA sequences based on hybridization techniques. This method involves a sample collection method whereby bacteria, viruses or other DNA containing species are collected and concentrated. This method also incorporates a sample preparation method that involves the liberation of the genetic components. After liberating the DNA, the sample is injected into a detection chip containing complementary DNA probes for the target of interest. In this manner, the device may contain multiple sets of probe molecules that each recognizes a single but different DNA sequence. This process ultimately involves the detection of hybridization products. In other embodiments of the present invention, the detection device includes a plurality of different groups of test structures, as shown in FIGS. 7-10.

In the collection phase, bacteria, viruses or other DNA containing samples are collected and concentrated. A plurality of collection methods will be used depending on the type of sample to be analyzed. Liquid samples will be collected by placing a constant volume of the liquid into a lysis buffer. Airborne samples can be collected by passing air over a filter for a constant time. The filter will be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

After sample collection and lysis, cell debris can be removed by precipitation or filtration. Ideally, the sample will be concentrated by filtration, which is more rapid and does not required special reagents. Samples will be forced through filters that will allow only the cellular material to pass through, trapping whole organisms and broken cell debris.

Here, the electrical conductivity of nucleic acid molecules is relied upon to transmit the electrical signal. Hans-Werner Fink and Christian Schoenenberger reported in Nature (1999), which is hereby incorporated by reference in its entirety, that DNA conducts electricity like a semiconductor. This flow of current can be sufficient to construct a simple switch, which will indicate whether or not a target nucleic acid molecule is present within a sample. Optionally, after hybridization of the target nucleic acid molecules to sets of capture probes, the nucleic acid molecules can be coated with a conductor, such as a metal. This technique is described in PCT Publication Nos. WO 99/04440, WO 99/57550, and WO 00/25136, U.S. Pat. No. 6,399,303, U.S. patent application Ser. Nos. 10/288,657 and 10/383,397, and U.S. Patent Publication Nos. US 20020182608 and US 20030040000, which are hereby incorporated by reference in their entirety. The coated nucleic acid molecule can then conduct electricity across the gap between the pair of probes, thus producing a detectable signal indicative of the presence of a target nucleic acid molecule. In order to increase the chances of capturing a target DNA of interest from a dilute and complex mixture of DNA sequences, multiple test structures can be placed within the test cartridge. These test structures can be used to detect the same target nucleic acid molecule, if present in a sample, a plurality of times or to detect different nucleic acid molecules, if present in a sample. In the latter case, the different probes can be designed to capture different target nucleic acid molecules from a single source (e.g. organism) to verify that that source is indeed present in a sample. Alternatively, the probe could be designed to capture target nucleic acid molecules from different sources (e.g. organisms) to permit a sample to be subjected to a battery of tests. These alternative strategies are particularly useful in analyzing a sample for pathogens. The advantage of this approach is to be able to overcome a low false positive rate by positively identifying the presence of any organism by use of statistics.

In carrying out the method of the present invention, a sample collection phase is initially carried out where bacteria, viruses, or other species are collected and concentrated. The target nucleic acid molecule whose sequence is to be determined is usually isolated from a tissue sample. If the target nucleic acid molecule is genomic, the sample may be from any tissue (except exclusively red blood cells). For example, whole blood, peripheral blood lymphocytes or PBMC, skin, hair, or semen are convenient sources of clinical samples. These sources are also suitable if the target is RNA. Blood and other body fluids are also a convenient source for isolating viral nucleic acids. If the target nucleic acid molecule is mRNA, the sample is obtained from a tissue in which the mRNA is expressed. If the target nucleic acid molecule in the sample is RNA, it may be reverse transcribed to DNA, but need not be converted to DNA in the present invention.

Further details of how to carry out the process of the present invention are set forth in U.S. Pat. No. 6,399,303 B1 to Connolly, which is hereby incorporated by reference in its entirety.

A plurality of collection methods can be used depending on the type of sample to be analyzed. Liquid samples can be collected by placing a constant volume of the liquid into a lysis buffer. Airborne samples can be collected by passing air over a filter for a constant time. The filter can be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

When whole cells, viruses, or other tissue samples are being analyzed, it is typically necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations such as denaturation of contaminating (DNA binding) proteins, purification, filtration, and desalting.

Liberation of nucleic acids from the sample cells or viruses, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea, to denature any contaminating and potentially interfering proteins. Generally, where chemical extraction and/or denaturation methods are used, the appropriate reagents may be incorporated within the extraction chamber, a separate accessible chamber, or externally introduced.

Alternatively, physical methods may be used to extract the nucleic acids and denature DNA binding proteins. U.S. Pat. No. 5,304,487, which is hereby incorporated by reference in its entirety, discusses the use of physical protrusions within microchannels or sharp edged particles within a chamber or channel to pierce cell membranes and extract their contents. More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysis/extraction, including, e.g., subjecting cells to ultrasonic agitation, or forcing cells through microgeometry apertures, thereby subjecting the cells to high shear stress resulting in rupture.

Following extraction, it is often desirable to separate the nucleic acids from other elements of the crude extract, e.g., denatured proteins, cell membrane particles, and the like. Removal of particulate matter is generally accomplished by filtration, flocculation, or the like. Ideally, the sample is concentrated by filtration, which is more rapid and does not require special reagents. A variety of filter types may be readily incorporated into the device. Samples can be forced through filters that will allow only the cellular material to pass through, trapping whole organisms and broken cell debris. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample. Suitable solid supports for nucleic acid binding include, e.g., diatomaceous earth, silica, or the like. Suitable gel exclusion media is also well known in the art and is commercially available from, e.g., Pharmacia and Sigma Chemical. This isolation and/or gel filtration/desalting may be carried out in an additional chamber, or alternatively, the particular chromatographic media may be incorporated in a channel or fluid passage leading to a subsequent reaction chamber.

The probes are preferably selected to bind with the target such that they have approximately the same melting temperature. This can be done by varying the lengths of the hybridization region. A-T rich regions may have longer target sequences, whereas G-C rich regions would have shorter target sequences.

Hybridization assays on substrate-bound oligonucleotide arrays involve a hybridization step and a detection step. In the hybridization step, the sample potentially containing the target and an isostabilizing agent, denaturing agent, or renaturation accelerant is brought into contact with the probes of the array and incubated at a temperature and for a time appropriate to allow hybridization between the target and any complementary probes.

Including a hybridization optimizing agent in the hybridization mixture significantly improves signal discrimination between perfectly matched targets and single-base mismatches. As used herein, the term “hybridization optimizing agent” refers to a composition that decreases hybridization between mismatched nucleic acid molecules, i.e., nucleic acid molecules whose sequences are not exactly complementary.

An isostabilizing agent is a composition that reduces the base-pair composition dependence of DNA thermal melting transitions. More particularly, the term refers to compounds that, in proper concentration, result in a differential melting temperature of no more than about 1° C. for double stranded DNA oligonucleotides composed of AT or GC, respectively. Isostabilizing agents preferably are used at a concentration between 1 M and 10 M, more preferably between 2 M and 6 M, most preferably between 4 M and 6 M, between 4 M and 10 M, and, optimally, at about 5 M. For example, a 5 M agent in 2×SSPE (Sodium Chloride/Sodium Phosphate/EDTA solution) is suitable. Betaines and lower tetraalkyl ammonium salts are examples of suitable isostabilizing agents.

Betaine (N,N,N,-trimethylglycine; (Rees et al., Biochem., (1993) 32:137-144), which is hereby incorporated by reference in its entirety) can eliminate the base pair composition dependence of DNA thermal stability. Unlike tetramethylammonium chloride (“TMACI”), betaine is zwitterionic at neutral pH and does not alter the polyelectrolyte behavior of nucleic acids while it does alter the composition-dependent stability of nucleic acids. Inclusion of betaine at about 5 M can lower the average hybridization signal, but increases the discrimination between matched and mismatched probes.

A denaturing agent is a compositions that lowers the melting temperature of double stranded nucleic acid molecules by interfering with hydrogen bonding between bases in a double-stranded nucleic acid or the hydration of nucleic acid molecules. Denaturing agents can be included in hybridization buffers at concentrations of about 1 M to about 6 M and, preferably, about 3 M to about 5.5 M.

Denaturing agents include formamide, formaldehyde, dimethylsulfoxide (“DMSO”), tetraethyl acetate, urea, guanidine thiocyanate (“GuSCN”), glycerol and chaotropic salts. As used herein, the term “chaotropic salt” refers to salts that function to disrupt van der Waal's attractions between atoms in nucleic acid molecules. Chaotropic salts include, for example, sodium trifluoroacetate, sodium tricholoroacetate, sodium perchlorate, and potassium thiocyanate.

A renaturation accelerant is a compound that increases the speed of renaturation of nucleic acids by at least 100-fold. They generally have relatively unstructured polymeric domains that weakly associate with nucleic acid molecules. Accelerants include heterogenous nuclear ribonucleoprotein (“hnRP”) A1 and cationic detergents such as, preferably, cetyltrimethylammonium bromide (“CTAB”) and dodecyl trimethylammonium bromide (“DTAB”), and, also, polylysine, spermine, spermidine, single stranded binding protein (“SSB”), phage T4 gene 32 protein, and a mixture of ammonium acetate and ethanol. Renaturation accelerants can be included in hybridization mixtures at concentrations of about 1 μM to about 10 mM and, preferably, 1 μM to about 1 mM. The CTAB buffers work well at concentrations as low as 0.1 mM.

Addition of small amounts of ionic detergents (such as N-lauroyl-sarkosine) to the hybridization buffers can also be useful. LiCl is preferred to NaCl. Hybridization can be at 20°-65° C., usually 37° C. to 45° C. for probes of about 14 nucleotides. Additional examples of hybridization conditions are provided in several sources, including: Sambrook et al., Molecular Cloning: A Laboratory Manual, 2nd Ed., Cold Spring Harbor, N.Y. (1989); and Berger and Kimmel, “Guide to Molecular Cloning Techniques,” Methods in Enzymology Volume 152, Academic Press, Inc., San Diego, Calif. (1987); Young and Davis, Proc. Natl. Acad. Sci. USA, 80:1194 (1983), which are hereby incorporated by reference in their entirety.

In addition to aqueous buffers, non-aqueous buffers may also be used. In particular, non-aqueous buffers which facilitate hybridization but have low electrical conductivity are preferred.

The sample and hybridization reagents are placed in contact with the array and incubated. Contact can take place in any suitable container, for example, a dish or a cell specially designed to hold the probe array and to allow introduction and removal of fluids. Generally, incubation will be at temperatures normally used for hybridization of nucleic acids, for example, between about 20° C. and about 75° C., e.g., about 25° C., about 30° C., about 35° C., about 40° C., about 45° C., about 50° C., about 55° C., about 60° C., or about 65° C. For probes longer than about 14 nucleotides, 37-45° C. is preferred. For shorter probes, 55-65° C. is preferred. More specific hybridization conditions can be calculated using formulae for determining the melting point of the hybridized region. Preferably, hybridization is carried out at a temperature at or between ten degrees below the melting temperature and the melting temperature. More preferred, hybridization is carried out at a temperature at or between five degrees below the melting temperature and the melting temperature. The target is incubated with the capture probes for a time sufficient to allow the desired level of hybridization between the target and any complementary capture probes. After incubation with the hybridization mixture, the electrically separated conductors are washed with the hybridization buffer, which also can include the hybridization optimizing agent. These agents can be included in the same range of amounts as for the hybridization step, or they can be eliminated altogether.

Details on how capture probes are attached to electrical conductors are set forth in U.S. patent application Ser. No. 10/288,657, which is hereby incorporated by reference in its entirety.

Various other methods exist for attaching the capture probes to the electrical conductors. For example, U.S. Pat. Nos. 5,861,242, 5,861,242, 5,856,174, 5,856,101, and 5,837,832, which are hereby incorporated by reference in their entirety, disclose a method where light is shone through a mask to activate functional (for oligonucleotides, typically an —OH) groups protected with a photo-removable protecting group on a surface of a solid support. After light activation, a nucleoside building block, itself protected with a photo-removable protecting group (at the 5′-OH), is coupled to the activated areas of the support. The process can be repeated, using different masks or mask orientations and building blocks, to place probes on a substrate.

Alternatively, new methods for the combinatorial chemical synthesis of peptide, polycarbamate, and oligonucleotide arrays have recently been reported (see Fodor et al., Science, 251:767-773 (1991); Cho et al., Science, 261:1303-1305 (1993); and Southern et al., Genomics 13:1008-10017 (1992), which are hereby incorporated by reference in their entirety). These arrays (see Fodor et al., Nature 364:555-556 (1993), which is hereby incorporated by reference in its entirety) harbor specific chemical compounds at precise locations in a high-density, information rich format, and are a powerful tool for the study of biological recognition processes.

Preferably, the probes are attached to the leads through spatially directed oligonucleotide synthesis. Spatially directed oligonucleotide synthesis may be carried out by any method of directing the synthesis of an oligonucleotide to a specific location on a substrate. Methods for spatially directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide which, itself, optionally has a protected active site if further nucleotide coupling is desired.

In one embodiment, the lead-bound oligonucleotides are synthesized at specific locations by light-directed oligonucleotide synthesis which is disclosed in U.S. Pat. No. 5,143,854, Published PCT Application Ser. No. WO 92/10092, and Published PCT Application Ser. No. WO 90/15070, which are hereby incorporated by reference in their entirety. In a basic strategy of this process, the surface of a solid support modified with linkers and photolabile protecting groups is illuminated through a photolithographic mask, yielding reactive hydroxyl groups in the illuminated regions. A 3′-O-phosphoramidite-activated deoxynucleoside (protected at the 5′-hydroxyl with a photolabile group) is then presented to the surface and coupling occurs at sites that were exposed to light. Following the optional capping of unreacted active sites and oxidation, the substrate is rinsed and the surface is illuminated through a second mask, to expose additional hydroxyl groups for coupling to the linker. A second 5′-protected, 3′-O-phosphoramidite-activated deoxynucleoside (C-X) is presented to the surface. The selective photodeprotection and coupling cycles are repeated until the desired set of probes are obtained. Photolabile groups are then optionally removed, and the sequence is, thereafter, optionally capped. Side chain protective groups, if present, are also removed. Since photolithography is used, the process can be miniaturized to specifically target leads in high densities on the support.

The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phospboramidite-activated deoxynucleoside. In this case, the oligonucleotide is synthesized in the 5′ to 3′ direction, which results in a free 5′ end.

The general process of removing protective groups by exposure to light, coupling nucleotides (optionally competent for further coupling) to the exposed active sites, and optionally capping unreacted sites is referred to herein as “light-directed nucleotide coupling.”

The probes may be targeted to the electrically separated conductors by using a chemical reaction for attaching the probe or nucleotide to the conductor which preferably binds the probe or nucleotide to the conductor rather than the support material. Alternatively, the probe or nucleotide may be targeted to the conductor by building up a charge on the conductor which electrostatically attracts the probe or nucleotide.

Nucleases can be used to remove probes which are attached to the wrong conductor. More particularly, a target nucleic acid molecule may be added to the probes. Targets which bind at both ends to probes, one end to each conductor, will have no free ends and will be resistant to exonuclease digestion. However, probes which are positioned so that the target cannot contact both conductors will be bound at only one end, leaving the molecule subject to digestion. Thus, improperly located probes can be removed while protecting the properly located probes. After the protease is removed or inactivated, the target nucleic acid molecule can be removed and the device is ready for use.

The capture probes can be formed from natural nucleotides, chemically modified nucleotides, or nucleotide analogs, as long as they have activated hydroxyl groups compatible with the linking chemistry. Such RNA or DNA analogs comprise but are not limited to 2′-O-alkyl sugar modifications, methylphosphonate, phosphorothioate, phosphorodithioate, formacetal, 3′-thioformacetal, sulfone, sulfamate, and nitroxide backbone modifications, amides, and analogs, where the base moieties have been modified. In addition, analogs of oligomers may be polymers in which the sugar moiety has been modified or replaced by another suitable moiety, resulting in polymers which include, but are not limited to, polyvinyl backbones (Pitha et al., “Preparation and Properties of Poly (I-vinylcytosine),” Biochim. Biophys. Acta, 204:381-8 (1970); Pitha et al., “Poly(1-vinyluracil): The Preparation and Interactions with Adenosine Derivatives,” Biochim. Biophys. Acta, 204:39-48 (1970), which are hereby incorporated by reference in their entirety), morpholino backbones (Summerton, et al., “Morpholino Antisense Oligomers: Design, Preparation, and Properties,” Antisense Nucleic Acid Drug Dev., 7:187-9 (1997), which is hereby incorporated by reference in its entirety) and peptide nucleic acid (PNA) analogs (Stein et al., “A Specificity Comparison of Four Antisense Types: Morpholino, 2′-O-methyl RNA, DNA, and Phosphorothioate DNA,” J. Antisense Nucleic Acid Drug Dev., 7:151-7 (1997); Egholm et al., “Peptide Nucleic Acids (PNA)-Oligonucleotide Analogues with an Achiral Peptide Backbone,” (1992); Faruqi et al., “Peptide Nucleic Acid-Targeted Mutagenesis of a Chromosomal Gene in Mouse Cells,” Proc. Natl. Acad. Sci. USA, 95:1398-403 (1998); Christensen et al., “Solid-Phase Synthesis of Peptide Nucleic Acids,” J. Pept. Sci., 1:175-83 (1995); Nielsen et al., “Peptide Nucleic Acid (PNA). A DNA Mimic with a Peptide Backbone,” Bioconjug. Chem., 5:3-7 (1994), which are hereby incorporated by reference in their entirety).

The capture probes can contain the following exemplary modifications: pendant moieties, such as, proteins (including, for example, nucleases, toxins, antibodies, signal peptides and poly-L-lysine); intercalators (e.g., acridine and psoralen), chelators (e.g., metals, radioactive metals, boron and oxidative metals), alkylators, and other modified linkages (e.g., alpha anomeric nucleic acids). Such analogs include various combinations of the above-mentioned modifications involving linkage groups and/or structural modifications of the sugar or base for the purpose of improving RNAseH-mediated destruction of the targeted RNA, binding affinity, nuclease resistance, and or target specificity.

The present invention can be used for numerous applications, such as detection of pathogens. For example, samples may be isolated from drinking water or food and rapidly screened for infectious organisms. The present invention may also be used for food and water testing. In recent times, there have been several large recalls of tainted meat products. The detection system of the present invention can be used for the in-process detection of pathogens in foods and the subsequent disposal of the contaminated materials. This could significantly improve food safety, prevent food borne illnesses and death, and avoid costly recalls. Capture probes that can identify common food borne pathogens, such as Salmonella and E. coli., could be designed for use within the food industry.

In yet another embodiment, the present invention can be used for real time detection of biological warfare agents. With the recent concerns of the use of biological weapons in a theater of war and in terrorist attacks, the device could be configured into a personal sensor for the combat soldier or into a remote sensor for advanced warnings of a biological threat. The devices which can be used to specifically identify the agent, can be coupled with a modem to send the information to another location. Mobile devices may also include a global positioning system to provide both location and pathogen information.

In yet another embodiment, the present invention may be used to identify an individual. A series of probes, of sufficient number to distinguish individuals with a high degree of reliability, are placed within the device. Various polymorphism sites are used. Preferentially, the device can determine the identity to a specificity of greater than one in one million, more preferred is a specificity of greater than one in one billion, even more preferred is a specificity of greater than one in ten billion.

### EXAMPLES

The following example is provided to illustrate embodiments of the present invention but are by no means intended to limit its scope.

### Example 1

### DNA Concentrator Card

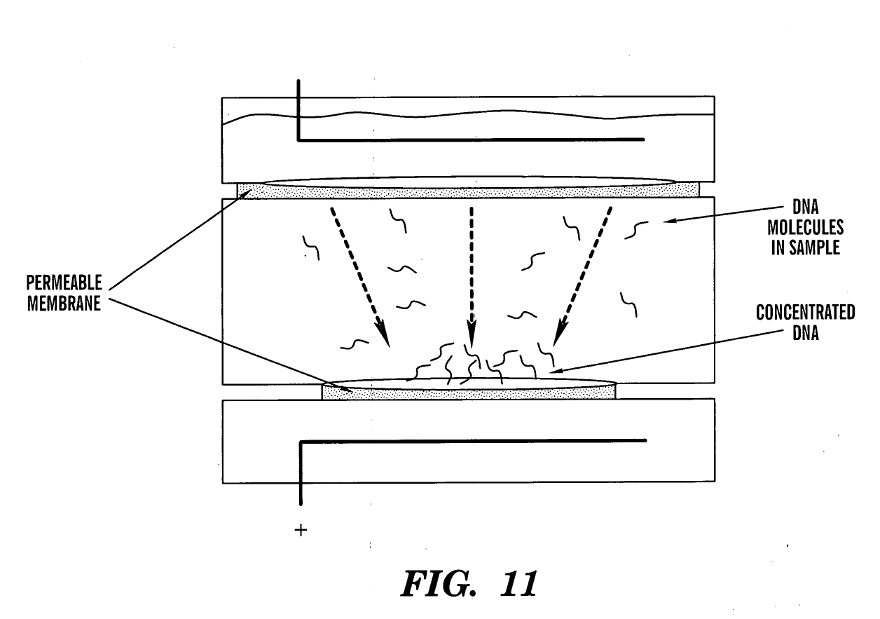

A DNA concentrator card that electrophoretically concentrates a dilute solution of dyed DNA from a large sample chamber down into a smaller collection channel was constructed as depicted in FIG. 11. When a voltage is applied to the opposite electrodes submerged in a buffer solution, an ionic current is established (the dashed arrows in FIG. 11 represent movement of negatively charged ions). In a compartment or chamber placed between the electrodes, which is permeable to the ionic flow, DNAs migrate towards the anode and the accumulate against the membrane wall of the container.

A dilute solution of an oligonucleotide, covalently coupled with the colored dye tamara™, was loaded into the sample chamber of the concentrator card. The upper and lower tank chambers, respectively housing the cathode and anode electrodes, were filled with a common electrophoresis buffer (e.g. Tris-Borate-EDTA). Voltages from 10 to 150 volts were applied, and the times for the colored DNA to concentrate into the collection channel were determined. At 120 volts, the faint red-colored DNA solution rapidly (in approximately 50 seconds) concentrated down into the collection channel. Lower voltages resulted in much slower DNA migration.

Although the invention has been described in detail for the purpose of illustration, it is understood that such detail is solely for that purpose, and variations can be made therein by those skilled in the art without departing from the spirit and scope of the invention that is defined by the following claims.

1. A device for concentrating and detecting target molecules in a flowing fluid sample, said device comprising:
a housing defining a passage through which a fluid sample flows;
a concentrating device positioned in the housing and comprising at least one electrode on a first side of the passage, wherein the at least one electrode on the first side of the passage has a polarity which electrostatically attracts target molecules in the flowing fluid sample; and
a detection device downstream of the concentrating device and comprising test structures having capture probes that are capable of specifically binding to the target molecules, if any, in the flowing fluid sample, wherein said test structures are positioned in the housing on the first side of the passage.

2. The device according to claim 1, further comprising:
at least one electrode in the concentrating device on a second side of the passage opposite the first side of the passage.

3. The device according to claim 2, wherein the at least one electrode on the first side of the passage and the at least one electrode on the second side of the passage are oppositely charged.

4. The device according to claim 2, wherein the at least one electrode on the first side of the passage and the at least one electrode on the second side of the passage are different in size.

5. The device according to claim 1, wherein the test structures comprise pairs of electrodes spaced apart by a gap with that gap being bridged by a conductor when the flowing fluid sample contains target molecules to which the capture probes are specific.

6. The device according to claim 5, wherein the capture probes are attached to the spaced apart electrical conductors such that a gap exists between the capture probes on the electrical conductors with that gap being bridged by a conductor when the flowing fluid sample contains target molecules to which the capture probes are specific.

7. The device according to claim 1, wherein the detection device comprises a plurality of different groups of test structures.

8. The device according to claim 1, wherein the capture probes are oligonucleotides.

9. The device according to claim 1, wherein the capture probes are peptide nucleic acid analogs.

10. The device according to claim 1, wherein the capture probes are antibodies.

11. The device according to claim 1, wherein the capture probes specifically bind to target molecules that are nucleic acid molecules.

12. The device according to claim 1, wherein the capture probes specifically bind to target molecules that are proteins.

13. The device according to claim 1 further comprising:
a flow reversing device, coupled to said housing, for reversing direction of fluid flowing through the passage, whereby fluid can be made to flow from the detection device to the concentrating device.

14. The device according to claim 1 further comprising:
a reconcentrating device positioned in the housing downstream of the detection device and comprising at least one electrode on the first side of the passage, wherein the at least one electrode on the first side of the passage of the reconcentrating device has a polarity which electrostatically attracts target molecules in the flowing fluid sample; and
a flow reversing device, coupled to said housing, for reversing direction of fluid flowing through the passage, whereby fluid can be made to flow from the reconcentrating device to the detection device.

15. The device according to claim 14 further comprising:
at least one electrode in the reconcentrating device on a second side of the passage opposite the first side of the passage with a polarity opposite that of the first electrode within the reconcentrating device to enhance movement of target molecules toward the electrodes on the first side.

16. The device according to claim 15, wherein the at least one electrode on the first side of the passage of the reconcentrating device and the at least one electrode on the second side of the reconcentrating device are oppositely charged.

17. The device according to claim 15, wherein the electrodes of the reconcentrating device are different in size.

18. The device according to claim 1, wherein the at least one electrode of the concentrating device is coated with a material that prevents the target molecules from contacting the electrode.

19. A method for concentrating and detecting target molecules in a flowing fluid sample, said method comprising:
providing a device comprising:
a housing defining a passage through which a fluid sample flows;
a concentrating device positioned in the housing and comprising at least one electrode on a first side of the passage, wherein the at least one electrode on the first side of the passage has a polarity which electrostatically attracts target molecules in the flowing fluid sample; and
a detection device downstream of the concentrating device and comprising test structures having capture probes that are capable of specifically binding to the target molecules, if any, in the flowing fluid sample, wherein said test structures are positioned in the housing on the first side of the passage;
introducing a sample containing target molecules into said device;
applying an electric field to the at least one electrode on the first side of the passage of the concentrating device under conditions effective to electrostatically attract and concentrate the target molecules, if any, in the flowing fluid sample near the at least one electrode on the first side of the passage of the concentrating device; and
permitting the concentrated target molecules in the flowing fluid sample to specifically bind to the capture probes of the detection device, wherein the presence of the target molecules in the flowing fluid sample is detected.

20. The method according to claim 19, wherein the concentrating device further comprises:
at least one electrode on a second side of the passage opposite the first side of the passage.

21. The method according to claim 20, wherein the at least one electrode on the first side of the passage and the at least one electrode on the second side of the passage are oppositely charged.

22. The method according to claim 20, wherein the at least one electrode on the first side of the passage and the at least one electrode on the second side of the passage are different in size.

23. The method according to claim 19, wherein the test structures comprise pairs of electrodes spaced apart by a gap with that gap being bridged by a conductor when the flowing fluid sample contains target molecules to which the capture probes are specific.

24. The method according to claim 23, wherein the capture probes are attached to electrical conductors such that a gap exists between the capture probes on the electrical conductors with that gap being bridged by a conductor when the flowing fluid sample contains target molecules to which the capture probes are specific.

25. The method according to claim 19, wherein the detection device comprises a plurality of different groups of test structures.

26. The method according to claim 19, wherein the capture probes are oligonucleotides.

27. The method according to claim 26, wherein the oligonucleotides are complementary to the genetic material of a pathogenic bacteria.

28. The method according to claim 27, wherein the pathogenic bacteria is a biowarfare agent.

29. The method according to claim 27, wherein the pathogenic bacteria is a food borne pathogen.

30. The method according to claim 26, wherein the oligonucleotides are complementary to the genetic material of a virus.

31. A method according to claim 26, wherein the oligonucleotides are complementary to the genetic material of a human.

32. A method according to claim 26, wherein the oligonucleotides have a sequence which is complementary to a sequence containing a polymorphism.

33. The method according to claim 19, wherein the capture robes are peptide nucleic acid analogs.

34. The method according to claim 19, wherein the capture probes are antibodies.

35. The method according to claim 19, wherein the target molecules are nucleic acid molecules.

36. The method according to claim 19, wherein the target molecules are proteins.

37. The method according to claim 19, wherein the device further comprises a flow reversing device, coupled to said housing, for reversing direction of fluid flowing through the passage, said method further comprising:
recycling the flowing fluid from the detection device to the concentrating device in order to reconcentrate target molecules in the flowing fluid sample that did not bind to the capture probes of the detection device.

38. The method according to claim 19, wherein the device further comprises:
a reconcentrating device positioned in the housing downstream of the detection device and comprising at least one electrode on the first side of the passage, wherein the at least one electrode on the first side of the passage of the reconcentrating device has a polarity which electrostatically attracts target molecules in the flowing fluid sample and a flow reversing device for reversing direction of fluid flowing through the passage, said method further comprising:
applying an electric field to the at least one electrode on the first side of the passage of the reconcentrating device under conditions effective to electrostatically attract and concentrate the target molecules in the flowing fluid sample; and
recycling the flowing fluid sample from the reconcentrating device to the detection device.

39. The method according to claim 38, wherein the device further comprises:
at least one electrode in the reconcentrating device on a second side opposite the first side of the passage with a polarity opposite the at least one electrode on the second side of the reconcentrating device, whereby target molecules in the flowing fluid sample within the reconcentrating device are electrostatically repelled by the electrodes on the second side toward the electrodes on the first side.

40. The method according to claim 39, wherein the at least two electrodes of the reconcentrating device are oppositely charged.

41. The method according to claim 39, wherein the at least two electrodes of the reconcentrating device are different in size.

42. The method according to claim 19, wherein the at least one electrode of the concentrating device is coated with a material that prevents the target molecules from contacting the electrode.

43. The method according to claim 19, wherein flow of the fluid sample is continuous.

44. The method according to claim 19, wherein flow of the fluid sample is pulsed.

45. The method according to claim 19, wherein the sample contains an additive that increases or decreases electrical mobility of the flowing fluid sample.

46. The method according to claim 45, wherein the additive is a salt, acid, or base.

47. The method according to claim 19, wherein the flowing fluid sample contains an additive that minimizes variations in viscosity.

48. The method according to claim 47, wherein the additive is a solution containing uncharged polymers.

49. The method according to claim 19, further comprising:
coating the capture probes as well as the target molecule bound to the capture probes with a conductive material after said permitting.

50. A method according to claim 49, wherein the conductive material is silver.

51. A method according to claim 49, wherein the conductive material is gold.
